(19) United States
(12) Patent Application Publication
(10) Pub. No.: US 2020/0321072 A1
(43) Pub. Date: Oct. 8, 2020

(54) A METHOD OF DETERMINING THE EFFECT OF MOLECULAR SUPPLEMENTS ON THE GUT MICROBIOME

(71) Applicant: Tata Chemicals Limited, Mumbai (IN)

(72) Inventor: Anirban Bhaduri, Mulshi, Pune (IN)

(21) Appl. No.: 16/956,243

(22) PCT Filed: Dec. 19, 2018
(86) PCT No.: PCT/IB2018/060320

§ 371 Date: Jun. 19, 2020

(30) Foreign Application Priority Data
Dec. 20, 2017 (IN) 201721045799

(51) Int. Cl. G16B 5/00; G16B 35/20; G16B 40/20; G06F 30/27; G06F 16/245; G06N 3/04; G06N 3/08

(52) U.S. Cl. CPC G16B 5/00; G16B 35/20; G16B 40/20; G06N 20/10; G06F 16/245; G06N 3/0418; G06N 3/08; G06F 30/27

(57) ABSTRACT

A method of determining effect of one or more molecular supplements on abundance of one or more subject microorganisms in one or more subject microbiome is disclosed. A device to determine effect of one or more molecular supplements on abundance of one or more subject microorganisms in one or more subject microbiome is also disclosed. Said device comprises of one or more input means, a memory, one or more processors, and a display device.

### FIELD OF INVENTION

The present disclosure relates to a method of determining effect of one or more molecular supplements on abundance of one or more subject microorganisms in one or more subject microbiome.

### BACKGROUND

Trillions of microorganisms are found in the human gut, and are collectively referred as the gut microbiome. Recent studies have estimated that the human gut hosts over 1000 unique micoorganisms or Operation Taxon Units (OTU). The gut microbiome contributes by processing molecular substrates and supplements, unutilized by the host. Recent evidences now substantiates that gut microbiome dysbiosis is associated with diseases such as diabetes, atherosclerosis, obesity and intestinal disorders

Given gut microbiome's role in health and wellness, efforts are being carried out towards its detailed characterization. Survey of gut microbiome provides us with an opportunity to understand how different molecular supplements within diet or otherwise impact microbiome of a subject. Knowledge of bacterial species in a particular gut environment, their predicted response to molecular supplements, in a background of bacterial communities, enables development of analytical engines to predict the composite response of the gut microbiome and its influence of health and wellness of the host subject.

Methods to predict response to food using microbiome profile are known. WO2015166489A2 discloses a method to predict response of a subject to food using microbiome profile. The method requires multi-dimensional data and partial microbiome data to report response.

Gut microbiome as a therapeutic and diagnostics marker is used for treatment and management of health conditions such as obesity, cardiovascular diseases, diabetes. Studies have correlated composition of an individual's microbiome, i.e. the number, identities, and relative abundance of microorganisms of the individual's microbiome with health conditions of the individual. US 20100172874 A1 discloses a method where gut microbiome was used as a biomarker and therapeutic target for energy harvesting, weight loss or gain, and/or obesity in a subject.

Current methods and system compute microbiome based host response to supplements. However, these personalized solutions are limiting since the response of molecular supplements are not accurate. This is because change in microbiome due to the molecular supplement and the composition of the complete microbiome, which is critical to compute the host response, is not considered.

Thus, there is a need to develop an effective method to determine the effect of molecular supplements on the gut microbiome. This would not only enable determining the effect of molecular supplements on the gut microbiome but also enable molecular supplements based guided nutritional design to carry out desired changes in the gut microbiome of an individual.

### SUMMARY

A method of determining effect of one or more molecular supplements on abundance of one or more subject microorganisms in one or more subject microbiome is disclosed. Said method comprises the steps of:
receiving, by a processor from one or more input means, an input data of the one or more molecular supplements and an abundance data of the one or more subject microorganisms in the one or more subject microbiome;
processing the abundance data of the one or more subject microorganisms in the one or more subject microbiome through a normalization process to record normalized abundance data;
extracting features from the normalized abundance data and creating a subject feature vector to represent the abundance data;
screening the subject feature vector against a knowledgebase stored in a memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on the abundance of one or more subject microorganisms in one or more subject microbiome;
identifying from the knowledgebase the feature vector which is similar to the subject feature vector and extracting the response model of said similar feature vector;
based on the response model, computing and determining the effect of the one or more molecular supplements on the abundance of one or more subject microorganisms in one or more subject microbiome; and
causing display of obtained information on a display device of a user.

A device to determine effect of one or more molecular supplements on abundance of one or more subject microorganisms in one or more subject microbiome is also disclosed. Said device comprises:

a. one or more input means;

b. a memory;

c. one or more processors; and

d. a display device;

wherein the one or more processors is configured to perform the steps of:
receiving from the one or more input means an input data of the one or more molecular supplements and an abundance data of the one or more subject microorganisms in one or more subject microbiome;
processing the abundance data of the one or more subject microorganisms in the one or more subject microbiome through a normalization process to record a normalized abundance data;
extracting features from the normalized abundance data and creating a subject feature vector to represent the input data;
screening the subject feature vector against a knowledgebase stored in the memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on abundance of one or more subject microorganisms in one or more subject microbiome;
identifying the feature vector from the knowledgebase which is similar to the subject feature vector and extracting the response model of said similar feature vector;
based on the response model, computing and determining the effect of the one or more molecular supplements on abundance of one or more subject microorganisms in one or more subject microbiome; and
causing display of obtained information on the display device of a user.

### BRIEF DESCRIPTION OF DRAWINGS

FIG. 1 illustrates the differential response of Bacteroides spp towards supplementation of 10 grams of Fructose oligosaccharide across 17 human subjects.

FIG. 2 illustrates schematic representation of the disclosed process, in accordance with an embodiment of the present invention.

FIG. 3 illustrates schematic representation of the disclosed process, in accordance with an embodiment of the present invention.

FIG. 4 illustrates pipeline adopted to collect and process fecal samples for obtaining the microbiome data, in accordance with an embodiment of the present invention.

FIG. 5 illustrates schematic representation of the knowledgebase, in accordance with an embodiment of the present invention

FIG. 6 illustrates pipeline employed to assess and validate the precision of the method, in accordance with an embodiment of the present invention

FIG. 7 reports the precision of the model across the 1000 iterative experiments, in accordance with an embodiment of the present invention.

FIG. 8 illustrates a neural network design, in accordance with an embodiment of the present invention.

### DETAILED DESCRIPTION

To promote an understanding of the principles of the invention, reference will be made to the embodiment and specific language will be used to describe the same. It will nevertheless be understood that no limitation of scope of the invention is thereby intended, such alterations and further modifications in the described product and such further applications of the principles of the inventions as disclosed therein being contemplated as would normally occur to one skilled in the art to which the invention relates.

In its broadest scope, the present disclosure relates to a method of determining effect of one or more molecular supplements on abundance of one or more subject microorganisms in one or more subject microbiome is disclosed. Said method comprises the steps of:
receiving, by a processor from one or more input means, an input data of the one or more molecular supplements and an abundance data of the one or more subject microorganisms in the one or more subject microbiome;
processing the abundance data of the one or more subject microorganisms in the one or more subject microbiome through a normalization process to record normalized abundance data;
extracting features from the normalized abundance data and creating a subject feature vector to represent the abundance data;
screening the subject feature vector against a knowledgebase stored in a memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on the abundance of one or more subject microorganisms in one or more subject microbiome;
identifying from the knowledgebase the feature vector which is similar to the subject feature vector and extracting the response model of said similar feature vector;
based on the response model, computing and determining the effect of the one or more molecular supplements on the abundance of one or more subject microorganisms in one or more subject microbiome; and
causing display of obtained information on a display device of a user.

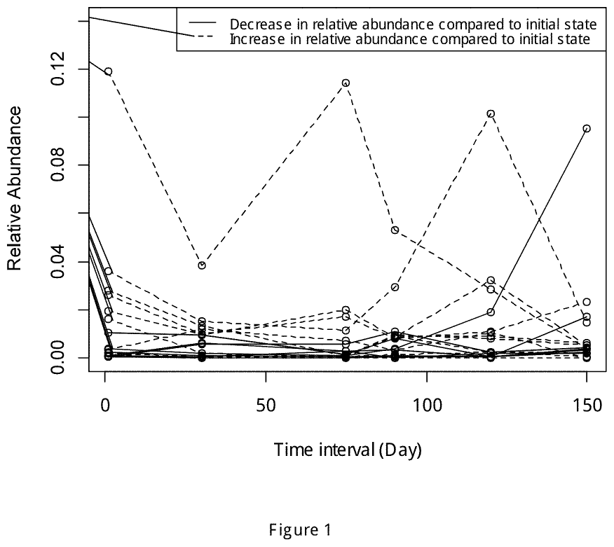

In accordance with an aspect, determining the effect of the one or more molecular supplements on the abundance of the one or more subject microorganisms in the one or more subject microbiome comprises determining, by the processor, relative and quantitative change in the abundance of the one or more subject microorganisms in the one or more subject microbiome when intervened by the one or more molecular supplements. As the response of individual microorganism to a molecular supplement is not consistent and is dependent on abundance and interaction of the microorganisms in the microbiome, the response of individual microorganism to a molecular supplement varies with the composition of the microbiome. The disclosed method determines the distinct effect of one or more molecular supplements on abundance of one or more subject microorganisms in one or more subject microbiome. In accordance with an exemplary embodiment, relative change of relative abundance of Bacteroides spp was studied on supplementation with Fructose oligosaccharide (FOS). As indicated in FIG. 1, it was observed that subjects sharing similar diet, physiological condition and supplemented by 10 grams of FOS reported a differential response of the relative abundance. The differential response is attributed to the difference in the initial microbiome composition in the individual subjects. The result further emphasizes the requirement of studying the complete microbiome to accurately report response of an organism towards a molecular supplement.

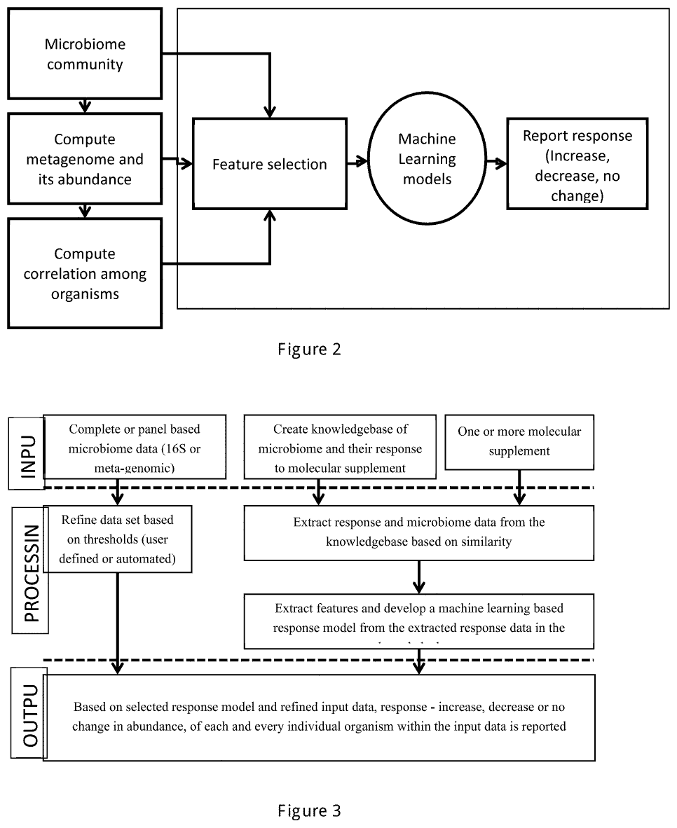

FIGS. 2 and 3 illustrate schematic representation of the disclosed process, in accordance with an embodiment of the present invention.

The reference microbiome refers to the microbiome of reference individuals. In accordance with an embodiment, one or more individuals may be the reference individuals. Preferably, the number of reference individuals is at least 3 for a given dosage of a given molecular supplement. The subject microbiome refers to the microbiome of a subject individual, on whose microbiome the effect of a molecular supplement has to be determined. The individual may be human or animal.

The data of the reference microbiome is obtained from fecal samples of the individuals, procured prior to the administration (i.e. pre-intervention) and after the administration (i.e. post-intervention) of the molecular supplement.

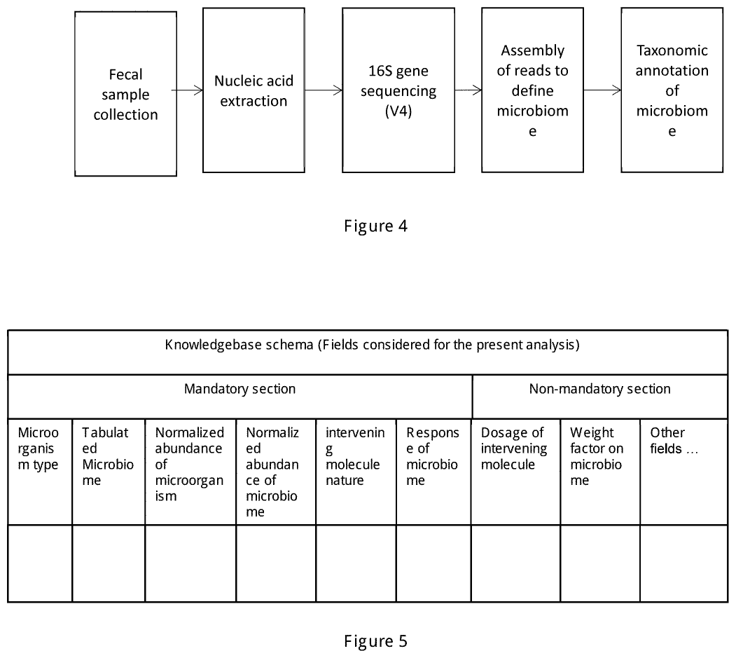

In accordance with an embodiment, the abundance data of the microbiome is recorded in terms of abundance count. The abundance count is obtained by a method selected from a group consisting of standard genomic sequencing, metagenomic sequencing, optical methods, and combinations thereof. In accordance with an exemplary embodiment, processing of the samples comprised of standard methods of nucleic acid extraction and 16S sequencing of the extracted DNA. An exemplary pipeline of sample processing to obtain and annotate the microbiome has been illustrated in FIG. 4.

In accordance with an embodiment, the abundance data of the one or more microorganisms in both the subject microbiome and the reference microbiome is processed and normalized by identifying and selecting the microorganisms which have the abundance count more than a threshold count. This threshold count may be a user or computational defined value based on the distribution of other microorganisms in the microbiome. Microorganisms reporting abundance count below the threshold count are considered as noise and dropped from the assessment. In accordance with an exemplary embodiment, the threshold count is 50.

In accordance with an embodiment, normalization of the abundance of the one or more microorganism is performed by applying known normalization algorithms. Said normalization algorithms, may include, but are not limited to transforming the data to relative abundance, transforming the data to odd score, or transforming the data to log odd score.

In accordance with an embodiment, the input data of the one or more molecular supplements comprises of chemical composition and dosage of the one or more molecular supplements.

The knowledgebase comprises of a plurality of feature vectors, each feature vector comprising data of a plurality of microorganisms present in the reference microbiome, abundance data of the plurality of microorganisms present in the reference microbiome before and after the administration of the one or more molecular supplement, and a response model to compute the effect of the one or more molecular supplements on the abundance of one or more subject microorganisms in one or more subject microbiome. In accordance with an embodiment, said data constitutes a mandatory section of the feature vectors. In accordance with an embodiment, the feature vectors listed in the knowledgebase further comprises of one or more data selected from a group consisting of dosage of the molecular supplement, localization of each microorganism, and weightage of each microorganism. In accordance with an embodiment, said features may constitute a non-mandatory section of the feature vectors. FIG. 5 illustrates an embodiment of schematic representation of the knowledgebase.

In accordance with an embodiment, the response of a microorganism to a molecular supplement is recorded in the knowledgebase in terms of fold change. The fold change refers to the ratio of post intervention normalized abundance to the pre intervention normalized abundance. In accordance with an embodiment, positive response was recorded, if the fold-change of the normalized abundance data was 1.2 or more. In accordance with an embodiment, negative response was recoded if the fold change was 0.8 or lower. Any other fold change was reported as neutral. In accordance with a related embodiment, response of one or more microorganism to one or more molecular supplement can be recorded as, but is not limited to, categorical or continuous data.

In accordance with an embodiment, the feature vector which is similar to the subject feature vector is identified on the basis of the subset of microbiome that establishes functional relationship with the one or more subject microorganism. Functional relationship is obtained based on the grouping members of the microbiome sharing similar response to the one or more subject microorganism. Functional relationship is quantified using similarity or distance scores. Relationship is established based on a user selected threshold score. Scoring of similarity or distance scoring is performed by, but is not limited to, correlation coefficient, Jaccard score, cosine, Euclidean distance etc.

In accordance with an embodiment, the response model is based on a supervised machine learning approach, including but not limited to neural network and support vector machine and a hybrid. In accordance with a preferred embodiment, the response model consists of neural network or its hybrid.

The present disclosure also relates to a device to determine effect of one or more molecular supplements on abundance of one or more subject microorganisms in one or more subject microbiome. Said device comprises:

a. one or more input means;

b. a memory;

c. one or more processors; and

d. a display device;

wherein the one or more processors is configured to perform the steps of:
receiving from the one or more input means an input data of the one or more molecular supplements and an abundance data of the one or more subject microorganisms in one or more subject microbiome;
processing the abundance data of the one or more subject microorganisms in the one or more subject microbiome through a normalization process to record a normalized abundance data;
extracting features from the normalized abundance data and creating a subject feature vector to represent the input data;
screening the subject feature vector against a knowledgebase stored in the memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on abundance of one or more subject microorganisms in one or more subject microbiome;
identifying the feature vector from the knowledgebase which is similar to the subject feature vector and extracting the response model of said similar feature vector;
based on the response model, computing and determining the effect of the one or more molecular supplements on abundance of one or more subject microorganisms in one or more subject microbiome; and
causing display of obtained information on the display device of a user.

In accordance with an embodiment, the input means, processor, memory and the display device may be any conventional input means, processor, memory and the display device respectively. In accordance with another embodiment, the processor, memory and display device may comprise multiple processors, memories and display devices respectively that may or may not be stored within the same physical housing.

### Specific Embodiments are Listed Below

A method of determining effect of one or more molecular supplements on abundance of one or more subject microorganisms in one or more subject microbiome, the method comprising the steps of:
receiving, by a processor from one or more input means, an input data of the one or more molecular supplements and an abundance data of the one or more subject microorganisms in the one or more subject microbiome;
processing the abundance data of the one or more subject microorganisms in the one or more subject microbiome through a normalization process to record normalized abundance data;
extracting features from the normalized abundance data and creating a subject feature vector to represent the abundance data;
screening the subject feature vector against a knowledgebase stored in a memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on the abundance of one or more subject microorganisms in one or more subject microbiome;
identifying from the knowledgebase the feature vector which is similar to the subject feature vector and extracting the response model of said similar feature vector;
based on the response model, computing and determining the effect of the one or more molecular supplements on the abundance of one or more subject microorganisms in one or more subject microbiome; and
causing display of obtained information on a display device of a user.

Such method, wherein determining the effect of the one or more molecular supplements on the abundance of the one or more subject microorganisms in the one or more subject microbiome comprises determining, by the processor, relative and quantitative change in the abundance of the one or more subject microorganisms in the one or more subject microbiome when intervened by the one or more molecular supplements.

Such method, wherein the input data of the one or more molecular supplements comprises of chemical composition and dosage of the one or more molecular supplements.

Such method, wherein the abundance data is recorded in terms of abundance count, the abundance count being obtained by a method selected from a group consisting of standard genomic sequencing, metagenomic sequencing, optical methods, and combination thereof.

Such method, wherein the abundance data of the one or more microorganisms in both the subject microbiome and the reference microbiome is processed and normalized by identifying and selecting the microorganisms which have the abundance count more than a threshold count, said threshold count being a user defined or computational defined value based on the distribution of other microorganisms in the microbiome.

Such method, wherein the feature vectors listed in the knowledgebase further comprises of one or more data selected from a group consisting of dosage of the molecular supplement, localization of each microorganism, and weightage of each microorganism.

Such method, wherein the response model is selected from a group consisting of neural network model and support vector machine model.

A device to determine effect of one or more molecular supplements on abundance of one or more subject microorganisms in one or more subject microbiome, the device comprising:

a. one or more input means;

b. a memory;

c. one or more processors; and

d. a display device;

wherein the one or more processors is configured to perform the steps of:
receiving from the one or more input means an input data of the one or more molecular supplements and an abundance data of the one or more subject microorganisms in one or more subject microbiome;
processing the abundance data of the one or more subject microorganisms in the one or more subject microbiome through a normalization process to record a normalized abundance data;
extracting features from the normalized abundance data and creating a subject feature vector to represent the input data;
screening the subject feature vector against a knowledgebase stored in the memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on abundance of one or more subject microorganisms in one or more subject microbiome;
identifying the feature vector from the knowledgebase which is similar to the subject feature vector and extracting the response model of said similar feature vector;
based on the response model, computing and determining the effect of the one or more molecular supplements on abundance of one or more subject microorganisms in one or more subject microbiome; and
causing display of obtained information on the display device of a user.

Such device, wherein determining the effect of the one or more molecular supplements on abundance of one or more subject microorganisms in one or more subject microbiome comprises determining, by the processor, relative and quantitative change in abundance of the one or more subject microorganisms in the one or more subject microbiome when intervened by the one or more molecular supplements.

### EXAMPLES

The following examples are provided to explain and illustrate the preferred embodiments of the process of the present invention and do not in any way limit the scope of the invention as described and claimed:

### Example 1

Molecular Supplement: Fructose oligosaccharides (FOS)
Dosage of Molecular Supplement: 10 grams/Day

### Threshold Count: 50

No. of Reference individuals: 20
Species: The 54 species whose response was predicted have been listed in Table 1, below.

TABLE 1

| Genus | Species |
|---|---|
| Prevotella | stercorea |
| 94otu9652 | 97otu2810 |
| Lactobacillus | ruminis |
| 94otu26993 | 97otu64685 |
| Bacteroides | 97otu98108 |
| Haemophilus | parainfluenzae |
| Faecalibacterium | prausnitzii |
| Catenibacterium | 97otu5040 |
| 94otu13460 | 97otu4334 |
| Sutterella | 97otu21533 |
| Sutterella | 97otu83458 |
| Lachnospira | 97otu7804 |
| 94otu13105 | 97otu730 |
| 94otu9878 | 97otu21191 |
| 94otu439 | 97otu6064 |
| 94otu11945 | 97otu43722 |
| Bacteroides | unclassified |
| Streptococcus | luteciae |
| 94otu3010 | 97otu3446 |
| Prevotella | unclassified |
| 94otu9391 | 97otu9097 |
| 94otu27202 | 97otu3079 |
| 94otu15100 | unclassified |
| 94otu17738 | 97otu48204 |
| 94otu811 | unclassified |
| Streptococcus | unclassified |
| 94otu10116 | 97otu91210 |
| 94otu15718 | 97otu21753 |
| Oscillospira | unclassified |
| Bifidobacterium | adolescentis |
| 94otu11726 | 97otu97516 |
| Lachnospira | unclassified |
| 94otu11484 | 97otu3021 |
| 94otu22540 | 97otu3206 |
| Roseburia | unclassified |
| Dialister | 97otu37193 |
| 94otu12870 | 97otu25481 |
| Unclassified | unclassified |
| 94otu28631 | 97otu31549 |
| Blautia | unclassified |
| 94otu20701 | 97otu71530 |
| 94otu11945 | 97otu18516 |
| 94otu13367 | 97otu2449 |
| Unclassified | unclassified |
| Lachnospira | 97otu55 |
| Roseburia | 97otu25366 |
| Roseburia | inulinivorans |
| Megasphaera | 97otu8385 |
| Coprococcus | eutactus |
| 94otu19924 | 97otu98 |
| Blautia | 97otu98146 |
| Ruminococcus | unclassified |
| 94otu22882 | 97otu85201 |
| Ruminococcus | callidus |
| Sutterella | stercoricanis |

As an exemplary embodiment to predict response of a microorganism to an intervening molecular composition, given a microbiome background a feed-forward neural network system based on multinomial log-linear models was developed.

### Data Collection

The study was conducted as per the pertinent requirements of the ICMR guidelines for Biomedical Research on Human Subjects, Good Clinical Practices for Clinical Research in India (http://www.icmr.nic.in/ethical_guidelines.pdf). The protocol was carried out in accordance with the approved guidelines, and was in agreement with Declaration of Helsinki principles.

Dataset used for the assessment comprised of microbiome extracted from fecal samples of 20 reference individuals. For each of reference individuals, 6 samples were obtained and processed. 3 samples were collected prior to intervention of the FOS while 3 were collected post intervention by FOS.

Processing of the samples comprised of standard methods of nucleic acid extraction and 16S sequencing of the extracted DNA. V4 region of the extracted 16S DNA gene were amplified and sequenced. Following sample processing using a standard pipeline (Second Genome pipeline), abundance of the various species within a sample was tabulated. The flowchart has been illustrated in FIG. 4.

### Knowledgebase Recording and Curation

Data from the samples of the 20 reference individuals were processed and was used to populate a knowledgebase. For each of the 20 reference individuals, average abundance of each member microorganism of the microbiome in the pre-intervention stage and the post intervention stage was recorded. Each member microorganism from a microbiome also called the reference microbiome was recorded as an entry. Only microorganism reporting an abundance of >50 counts were considered for recording. Microorganisms reporting a count below 50 was considered as noise and dropped. Against each entry, normalized average abundance for the pre and post intervention by FOS (fructose oligosaccharide), the members of the reference microbiome and their average normalized abundance, response to the FOS, as fold change were also recorded.

Normalized average abundance of the entered microorganism for pre and post intervention by FOS was computed by normalizing its average abundance against the total abundance of all member microorganisms for the entered microbiome.

Following the above mentioned approach each member of the reference microbiome was also normalized and the normalized average abundance of the reference microbiome against an entered microorganism was tabulated in the knowledgebase.

Response of the microorganism towards the intervening molecular composition was of 3 types, positive negative and neutral. The knowledgebase recorded positive response against the entered microorganism if the fold-change of the normalized abundance data was 1.2 or more. Negative response was recoded if the fold change was 0.8 or lower. Any other fold change was reported as neutral.

### Machine Learning Approach Development

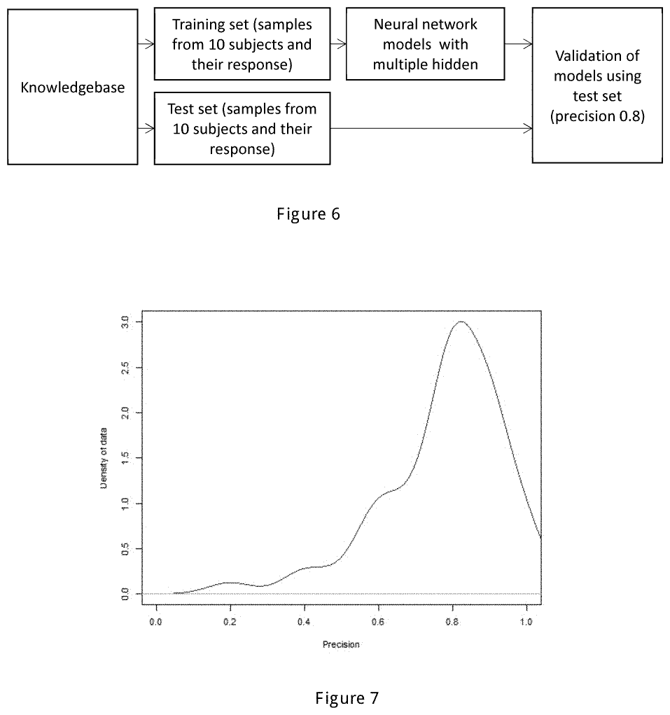

A feed-forward neural network system with a single hidden layer and multinomial log-linear models was used to develop a machine learning method. Data sets from the current knowledgebase were divided into two equal halves for the assessment. One of the half was used to train and develop the machine learning model. This set was referred as the training set. The other half was used to validate the developed models. This half was referred as the test set. The distribution of the data into training and test was performed randomly and iterated for assessing the robustness of the approach. Input training and testing data included the understudy microorganism with normalized abundance, normalized microbiome from where the understudy microorganism is obtained. The machine learning models were trained using the response tabulated in the knowledgebase. The flow chart shown in FIG. 6 summarizes the processing steps.

### Assessment of the Model Quality

In order to evaluate the robustness of the mentioned method, 1000 random training data sets were extracted from the curated knowledgebase and neural network models were developed. In parallel, test data set was also extracted and validated against the developed models. Assessment of the approach was based on the precision of the models to predict response of the test dataset. Average precision of the method obtained from the unbiased sampling experiment reported a value of ˜0.8. FIG. 7 reports the density plot of the precision of the model across 1000 iterative experiments.

### Example 2: Prediction of Response of Lactobacillus Ruminis

### Molecular Supplement: FOS

Dosage: 10 g/Day

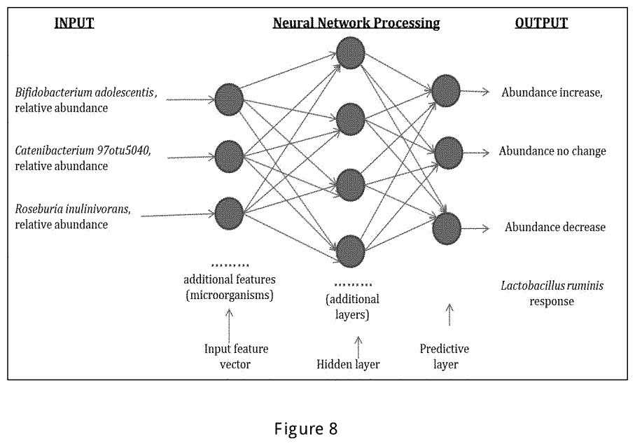

Herein, response of Lactobacillus ruminis towards 10 grams of FOS is reported. A knowledgebase with similar structure as in Example 1 was used. The response reported is increase in relative abundance, no change in relative abundance or relative decrease in abundance of Lactobacillus ruminis when intervened with 10 grams of FOS as a daily dosage in the background of a given microbiome. The complete microbiome is represented by 53 features (53 different microorganisms). The complete model comprised of feed-forward neural network system with a single hidden layer and multinomial log-linear model. For simplicity of representation the top three features (Bifidobacterium adolescentis, Catenibacterium 97otu5040 and Roseburia inulinivorans) are used to show the structure of the neural models and has been illustrated in FIG. 8. Weights of the models are not reported in the abridged figure. Complete model for of Lactobacillus ruminis is represented through 177 connections.

Table 2 summarizes the experimental response and the prediction derived from the model. Given relative abundance of Bifidobacterium adolescentis, Catenibacterium 97otu5040 and Roseburia inulinivorans abundance change of Lactobacillus ruminis on intervention with FOS is predicted in 10 samples. Prediction is compared to experimental observations. Table 2 suggests that with 2 exceptions the method accurately predict response of FOS (Precision 0.8).

TABLE 2

| | Input (Relative abundance) | | | Lactobacillus ruminis | |
|---|---|---|---|---|---|
| | Bifidobacterium | Catenibacterium | Roseburia | Response | |
| Sample | adolescentis | 97otu5040 | inulinivorans | Prediction | Experiment |
| 1 | 8.80e−03 | 2.84e−02 | 8.27e−03 | Increase | Increase |
| 2 | 3.82e−02 | 9.63e−03 | 6.76e−05 | Decrease | Decrease |
| 3 | 1.06e−01 | 7.21e−05 | 2.42e−03 | Increase | Decrease |
| 4 | 4.29e−02 | 6.58e−02 | 1.10e−02 | Increase | Increase |
| 5 | 8.78e−02 | 4.33e−02 | 4.99e−03 | Increase | Decrease |
| 6 | 2.94e−02 | 2.42e−02 | 6.02e−03 | Decrease | Decrease |
| 7 | 1.17e−02 | 1.34e−05 | 1.02e−02 | Increase | Increase |
| 8 | 3.93e−02 | 9.27e−03 | 1.14e−03 | Increase | Increase |
| 9 | 6.63e−02 | 1.95e−02 | 6.99e−03 | Increase | Decrease |
| 10 | 3.70e−02 | 2.52e−02 | 1.03e−05 | Increase | Increase |

### INDUSTRIAL APPLICABILITY

The present disclosure relates to a method of predicting the effect of one or more molecular supplement on the microbiome of a subject individual. Said molecular supplement may include, but is not limited to, one or more of prebiotics, active pharmaceutical ingredients (APIs), dietary supplements and the like.

As the response of individual microorganism to a molecular supplement is not consistent and is dependent on interaction of the microorganisms among themselves in the microbiome, the disclosed method determines the specific effect of one or more molecular supplements on abundance of one or more subject microorganisms in one or more subject microbiome.

The disclosed method may be used to design nutraceutical compositions for a particular individual to achieve desired effect on the microbiome of the individual. The disclosed method may be applied for any molecular supplement and any dosage thereof.

The present invention may be employed for prediction of microbiome of humans as well as animals.

1. A method of determining effect of one or more molecular supplements on abundance of one or more subject microorganisms in one or more subject microbiome, the method comprising the steps of:
receiving, by a processor from one or more input means, an input data of the one or more molecular supplements and an abundance data of the one or more subject microorganisms in the one or more subject microbiome;
processing the abundance data of the one or more subject microorganisms in the one or more subject microbiome through a normalization process to record normalized abundance data;
extracting features from the normalized abundance data and creating a subject feature vector to represent the abundance data;
screening the subject feature vector against a knowledgebase stored in a memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on the abundance of one or more subject microorganisms in one or more subject microbiome;
identifying from the knowledgebase the feature vector which is similar to the subject feature vector and extracting the response model of said similar feature vector;
based on the response model, computing and determining the effect of the one or more molecular supplements on the abundance of one or more subject microorganisms in one or more subject microbiome; and
causing display of obtained information on a display device of a user.

2. The method as claimed in claim 1, wherein determining the effect of the one or more molecular supplements on the abundance of the one or more subject microorganisms in the one or more subject microbiome comprises determining, by the processor, relative and quantitative change in the abundance of the one or more subject microorganisms in the one or more subject microbiome when intervened by the one or more molecular supplements.

3. The method as claimed in claim 1, wherein the input data of the one or more molecular supplements comprises of chemical composition and dosage of the one or more molecular supplements.

4. The method as claimed in claim 1, wherein the abundance data is recorded in terms of abundance count, the abundance count being obtained by a method selected from a group consisting of standard genomic sequencing, metagenomic sequencing, optical methods, and combination thereof.

5. The method as claimed in claim 1, wherein the abundance data of the one or more microorganisms in both the subject microbiome and the reference microbiome is processed and normalized by identifying and selecting the microorganisms which have the abundance count more than a threshold count, said threshold count being a user defined or computational defined value based on the distribution of other microorganisms in the microbiome.

6. The method as claimed in claim 1, wherein the feature vectors listed in the knowledgebase further comprises of one or more data selected from a group consisting of dosage of the molecular supplement, localization of each microorganism, and weightage of each microorganism.

7. The method as claimed in claim 1, wherein the response model is selected from a group consisting of neural network model and support vector machine model.

8. A device to determine effect of one or more molecular supplements on abundance of one or more subject microorganisms in one or more subject microbiome, the device comprising:
a. one or more input means;
b. a memory;
c. one or more processors; and
d. a display device;
wherein the one or more processors is configured to perform the steps of:
receiving from the one or more input means an input data of the one or more molecular supplements and an abundance data of the one or more subject microorganisms in one or more subject microbiome;
processing the abundance data of the one or more subject microorganisms in the one or more subject microbiome through a normalization process to record a normalized abundance data;
extracting features from the normalized abundance data and creating a subject feature vector to represent the input data;
screening the subject feature vector against a knowledgebase stored in the memory, the knowledgebase comprising a plurality of feature vectors, each feature vector comprising data of a plurality of microorganisms present in a reference microbiome, abundance data of the plurality of microorganisms present in the reference microbiome before and after the administration of the one or more molecular supplement, and a response model to compute the effect of the one or more molecular supplements on abundance of one or more subject microorganisms in one or more subject microbiome;
identifying the feature vector from the knowledgebase which is similar to the subject feature vector and extracting the response model of said similar feature vector;
based on the response model, computing and determining the effect of the one or more molecular supplements on abundance of one or more subject microorganisms in one or more subject microbiome; and
causing display of obtained information on the display device of a user.

9. The device as claimed in claim 8, wherein determining the effect of the one or more molecular supplements on abundance of one or more subject microorganisms in one or more subject microbiome comprises determining, by the processor, relative and quantitative change in abundance of the one or more subject microorganisms in the one or more subject microbiome when intervened by the one or more molecular supplements.
